(19) United States
(12) Patent Application Publication
(10) Pub. No.: US 2025/0331893 A1
(43) Pub. Date: Oct. 30, 2025

(54) DEVICES AND METHODS FOR PALPATING AND PENETRATING A JOINT CAPSULE

(71) Applicant: Stryker Corporation, Portage, MI (US)

(72) Inventors: William KAISER, Campbell, CA (US); Nicolas OLIVARES, Centennial, CO (US); Harold SAMPSON, Denver, CO (US)

(73) Assignee: Stryker Corporation, Portage, MI (US)

(21) Appl. No.: 19/190,465

(22) Filed: Apr. 25, 2025

Related U.S. Application Data
(60) Provisional application No. 63/639,563, filed on Apr. 26, 2024.

(51) Int. Cl. A61B 17/34

(52) U.S. Cl. CPC A61B 17/3472

(57) ABSTRACT

A device for palpating and penetrating a joint capsule of a subject includes a shaft having a distal tip configured for palpating the joint capsule of the subject; a needle disposed within the shaft and configured to penetrate the joint capsule of the subject; and a handle coupled to the shaft and comprising a slider configured to translate the needle between a retracted state in which a distal end of the needle is retracted within the shaft and a fully deployed state in which the distal end of the needle fully extends from the distal tip of the shaft.

### CROSS-REFERENCE TO RELATED APPLICATIONS

This application claims the benefit of U.S. Provisional Application No. 63/639,563, filed Apr. 26, 2024, the entire contents of which are hereby incorporated by reference herein.

### FIELD

This disclosure relates generally to arthroscopic surgery, and more specifically, to methods and devices for penetrating a joint capsule to access a joint.

### BACKGROUND

A joint capsule is made up of tough, fibrous tissue that can be difficult to penetrate. Because of this toughness, it can be difficult to control the insertion of instruments through it, which often leads to uncontrolled entry into the joint space and damage to the tissues therein (e.g., articular cartilage and the labrum). Moreover, in joints such as the hip, the thickness of the muscles, skin, and fat surrounding the joint can be significant, which introduces additional obstacles to accessing and penetrating the joint capsule to access the joint.

Identifying a suitable location for penetrating a joint capsule can be challenging. Surgeons typically rely on a limited number of anatomical landmarks proximate to the joint capsule. However, effective use of these few anatomical landmarks varies based on experience and training. Surgeons can also use fluoroscopy to visualize the joint and help identify a suitable location for joint capsule penetration. However, fluoroscopy is two-dimensional and thus provides limited information to the surgeon who is attempting to ascertain a three-dimensional position for penetrating the joint capsule.

Existing methods for penetrating a joint capsule use long, thin needles (e.g., spine needles) to initially penetrate the joint capsule. Because spine needles are flimsy, they provide minimal tactile feedback to the user during placement and are difficult to reposition if inserted at the wrong trajectory in the joint capsule. Spine needles are also very sharp, which increases the risk of damage to tissue in the joint space if improperly inserted through the joint capsule. Once the joint capsule is penetrated with the spine needle, a flow cannula having a diameter that is significantly larger than that of the needle is inserted into the path in the joint capsule created by the needle. Inserting the flow cannula in this manner is often challenging because, in combination with the toughness of the joint capsule, the large diameter of the cannula prevents the cannula from being inserted through the joint capsule. The surgeon can try to force the cannula through the joint capsule along the path of the needle, but this force can cause undesired trauma to the joint capsule. Alternatively, in some instances, a set of increasingly larger sized dilators are used to carefully dilate the joint capsule prior to inserting the cannula through the capsule. However, this approach adds several tedious steps to the procedure and necessitates several different instruments that must fit well within each other.

### SUMMARY

According to an aspect, described herein are devices and methods for palpating and penetrating a joint capsule of a subject. A palpating device is configured for a user to palpate the joint capsule to determine a suitable location for penetrating the joint capsule. The palpating device can include a needle that can be in a retracted state during palpation and can be translated to a deployed state to penetrate the joint capsule. In some examples, the palpating device may be configured to extend the needle in a partially deployed state in which the needle can partially penetrate the joint capsule, which may be useful for a user to assess the position of the palpating device prior to fully penetrating the joint capsule. The palpating device may include or be fitted with a cannula that can be inserted into the joint capsule through the path in the joint capsule created by the palpating device to establish a portal to the joint space for further procedures on the joint. By palpating the joint capsule using the palpating devices described herein prior to penetrating the joint capsule, cannula placement in the joint capsule can be optimized, and damage to surrounding tissues in the joint can be minimized. Moreover, because the palpating devices provided herein are used to both palpate and penetrate the joint capsule, joint access procedures can be simplified and shortened by providing one device that can perform both operations.

According to an aspect, a device for palpating and penetrating a joint capsule of a subject includes a shaft having a distal tip configured for palpating the joint capsule of the subject; a needle disposed within the shaft and configured to penetrate the joint capsule of the subject; and a handle coupled to the shaft and comprising a slider configured to translate the needle between a retracted state in which a distal end of the needle is retracted within the shaft and a fully deployed state in which the distal end of the needle fully extends from the distal tip of the shaft.

The slider may removably lock into place with the handle when the needle is in the retracted state and the fully deployed state. The distal end of the needle may be retracted at least 1 mm from the distal tip of the shaft in the retracted state. The distal end of the needle may extend between a length that is greater than 10 mm to 30 mm from the distal tip of the shaft in the fully deployed state. The slider may be configured to translate the needle between the retracted state and a partially deployed state in which the distal end of the needle partially extends from the distal tip of the shaft. The slider may removably lock into place with the handle when the needle is in the partially deployed state. The distal end of the needle may extend between a length that is greater than 0 mm to 10 mm from the distal tip of the shaft in the partially deployed state. Each of the needle, the handle, and the slider may include a guidewire lumen. The handle may be configured to removably attach to a cannula configured to receive the shaft. A leading tip of the needle may be aligned with a side of the handle comprising the slider for orienting the leading tip of the needle. The distal tip may include a blunt distal-most end oriented orthogonally to a longitudinal axis of the shaft. The blunt distal-most end may be configured to facilitate palpating the joint capsule without penetrating the joint capsule. A diameter of the distal tip of the shaft may taper to a minimum diameter at the blunt distal-most end to facilitate inserting a distal portion of the shaft through the joint capsule. The shaft may include a continuous curve to the distal tip of the shaft to facilitate inserting a distal portion of the shaft through the joint capsule.

According to an aspect, a method for palpating and penetrating a joint capsule of a subject includes inserting a distal portion of a palpating device into a target site to contact the joint capsule of the subject; palpating the joint capsule using a distal tip of the palpating device to determine an appropriate position for penetrating the joint capsule; and upon determination of the appropriate position, extending a needle of the palpating device to penetrate the joint capsule.

The palpating device can include a shaft having the distal tip of the palpating device for palpating the joint capsule, and the needle of the palpating device is disposed within the shaft. The palpating device can include a handle coupled to the shaft and comprising a slider, such that extending the needle includes moving the slider to cause the needle to translate between a retracted state in which a distal end of the needle is retracted within the shaft and a fully deployed state in which the distal end of the needle fully extends from the distal tip of the shaft. The distal end of the needle may be retracted at least 1 mm from the distal tip of the shaft in the retracted state. The distal end of the needle may extend between a length that is greater than 10 mm to 30 mm from the distal tip of the shaft in the fully deployed state. The slider may removably lock into place with the handle in the retracted state and the fully deployed state of the needle. Extending the needle may include moving the slider to cause the needle to translate between the retracted state and a partially deployed state in which the distal end of the needle partially extends from the distal tip of the shaft to partially penetrate joint capsule. Extending the needle may include moving the slider to cause the needle to translate between the partially deployed state and the fully deployed state to fully penetrate the joint capsule. The distal end of the needle may extend to a length that is greater than 0 mm to 10 mm from the distal tip of the shaft in the partially deployed state. The slider may removably lock into place with the handle in the partially deployed state.

The method may include, prior to fully extending the needle of the palpating device to penetrate the joint capsule, imaging the target site having the palpating device inserted therein to determine whether the palpating device is in the appropriate position for penetrating the joint capsule. The method may include, after penetrating the joint capsule with the needle, inserting a guidewire through a lumen of the palpating device and through the joint capsule. The method may include, after penetrating the joint capsule with the needle, retracting the needle from the joint capsule. The method may include, after penetrating the joint capsule, inserting the distal portion of the palpating device through the joint capsule. The palpating device may include a continuous curve to the distal tip of the shaft to facilitate inserting the distal portion of the palpating device through the joint capsule. Extending the needle to penetrate the joint capsule may include partially extending the needle to partially penetrate the joint capsule, and the method may include, after partially penetrating the joint capsule, fully extending the needle to fully penetrate the joint capsule. Inserting the distal portion of the palpating device into the target site may include inserting a cannula attached to the distal portion of the palpating device into the target site. Extending the needle of the palpating device may include orienting a leading tip of the needle that is aligned with a side of the handle that includes the slider. The distal tip of the palpating device may include a blunt distal-most end for palpating the joint capsule without penetrating the joint capsule.

According to an aspect, a cannula for viewing a surgical site includes a shaft having an angled distal end. The angled distal end of the cannula can correspond to an angled distal end of an endoscope. When inserted into the cannula, the angled distal end of the endoscope can stand proud of the angled distal end of the cannula.

It will be appreciated that any of the variations, aspects, features, and options described in view of the systems apply equally to the methods and vice versa. It will also be clear that any one or more of the above variations, aspects, features, and options can be combined.

### BRIEF DESCRIPTION OF THE FIGURES

The invention will now be described, by way of example only, with reference to the accompanying drawings, in which:

FIG. 1A shows a side view of an exemplary palpating device with a needle of the palpating device in a retracted state;

FIG. 1B shows a partial view of the exemplary palpating device of FIG. 1A with the needle in a partially deployed state;

FIG. 1C shows a partial view of the exemplary palpating device of FIGS. 1A-1B with the needle in a fully deployed state;

FIG. 2 shows a handle of an exemplary palpating device;

FIG. 3A shows a distal portion of an exemplary palpating device with the needle of the palpating device in a retracted state;

FIG. 3B shows the distal portion of the exemplary palpating device of FIG. 3A with the needle in the partially deployed state;

FIG. 3C shows the distal portion of the exemplary palpating device of FIGS. 3A-3B with the needle in the fully deployed state;

FIG. 4A shows an exploded view of the exemplary palpating device of FIGS. 1A-1C and an exemplary cannula configured to removably attach to the exemplary palpating device;

FIG. 4B shows a side view of the exemplary palpating device of FIGS. 1A-1C with the shaft of the palpating device received by the exemplary cannula;

FIG. 5A shows a side view of an exemplary palpating device having a decoupled needle, the decoupled needle assembled with the remainder of the palpating device;

FIG. 5B shows a side view of the exemplary palpating device of FIG. 5A, with the decoupled needle disassembled from the remainder of the palpating device;

FIG. 5C shows a side view of the exemplary palpating device of FIGS. 5A-5B;

FIG. 5D shows a top view of the exemplary palpating device of FIGS. 5A-5C;

FIG. 6 shows a side view of an exemplary palpating device having a threaded mechanism for translating a needle through a shaft of the palpating device;

FIG. 7A shows a diagram of an exemplary palpating device positioned at a location of the joint capsule overlying bony anatomy;

FIG. 7B shows a diagram of the exemplary palpating device pressed into a portion of the joint capsule that is overlying the joint space;

FIG. 7C shows a diagram of a needle of the exemplary palpating device partially penetrating the joint capsule;

FIG. 7D shows a diagram of the needle of the exemplary palpating device fully penetrating the joint capsule;

FIG. 7E shows a diagram of a distal portion of the exemplary palpating device inserted through the joint capsule and into the joint space;

FIG. 8A shows a prospective view of a distal portion of an exemplary cannula having an exemplary endoscope disposed therein; and

FIG. 8B shows a side view of the distal portion of the cannula and endoscope of FIG. 8A.

### DETAILED DESCRIPTION

Reference will now be made in detail to implementations and examples of various aspects and variations of systems and methods described herein. Although several exemplary variations of the systems and methods are described herein, other variations of the systems and methods may include aspects of the systems and methods described herein combined in any suitable manner having combinations of all or some of the aspects described.

According to an aspect, devices and methods described herein can be used for palpating and penetrating a joint capsule of a subject. Palpating devices described herein can simplify procedures for accessing a joint by providing a single device for both palpating and penetrating the joint capsule of a subject. An exemplary palpating device includes a distal tip configured for palpating the joint capsule to determine a suitable location for penetrating the joint capsule. The palpating device can include a needle translatable between a retracted state in which the palpating device can be used for palpating the joint capsule and at least one deployed state for penetrating the joint capsule.

The palpating device may removably lock the needle into place in each of the states, which may ensure a more controlled, safe penetration of the joint capsule, and in turn reduce any risk of damage to the tissue surrounding the joint (e.g., articular cartilage or labrum). After the joint capsule is penetrated with the needle, the palpating device can be inserted into the joint capsule, through the path in the joint capsule created by the needle. Optionally, after penetrating the joint capsule, a guidewire can be inserted through a lumen of the palpating device and through the joint capsule (e.g., to allow the needle to be retracted into the palpating device for safety and/or to guide placement of a cannula through the joint capsule and into the joint space). The distal portion of the palpating device can be shaped to ensure the palpating device can be smoothly inserted through the joint capsule, thus minimizing any trauma to the joint capsule. A cannula, either attached to the shaft of the palpating device or separate from the palpating device, may be inserted through the path created by the palpating device in the joint capsule to establish a portal to the joint space. Thus, by providing a device that enables palpation of the joint capsule prior to penetrating the joint capsule, cannula placement in the joint space for further operations on the joint of the subject can be optimized.

In the following description of the various examples, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

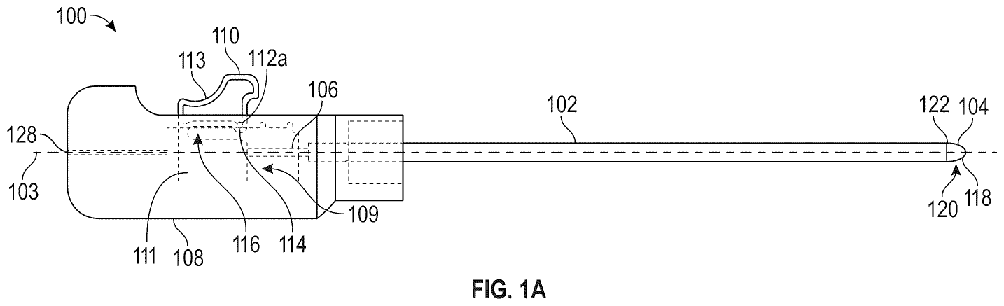

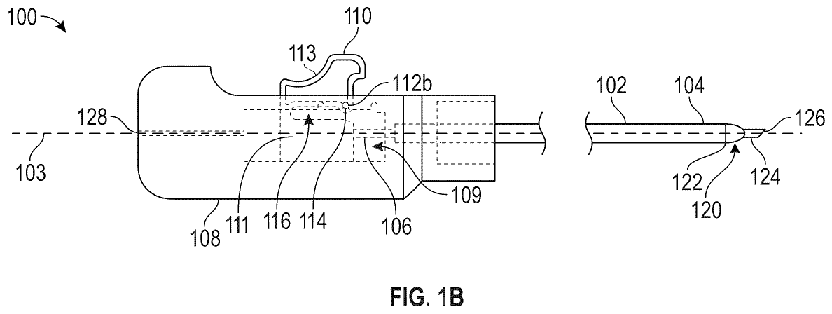

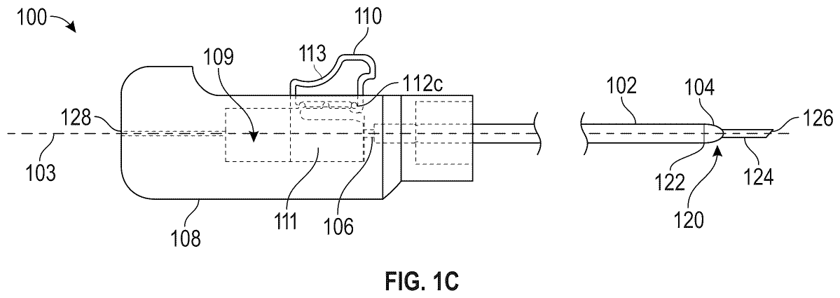

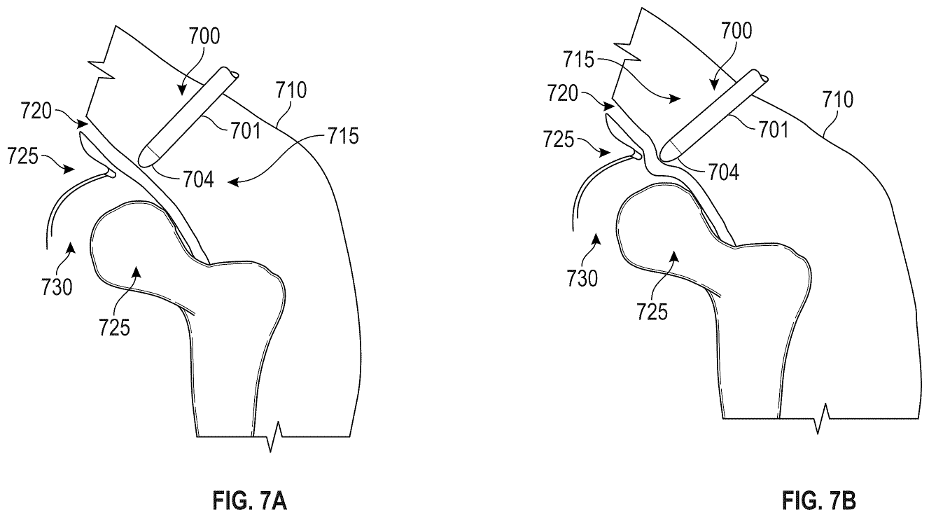

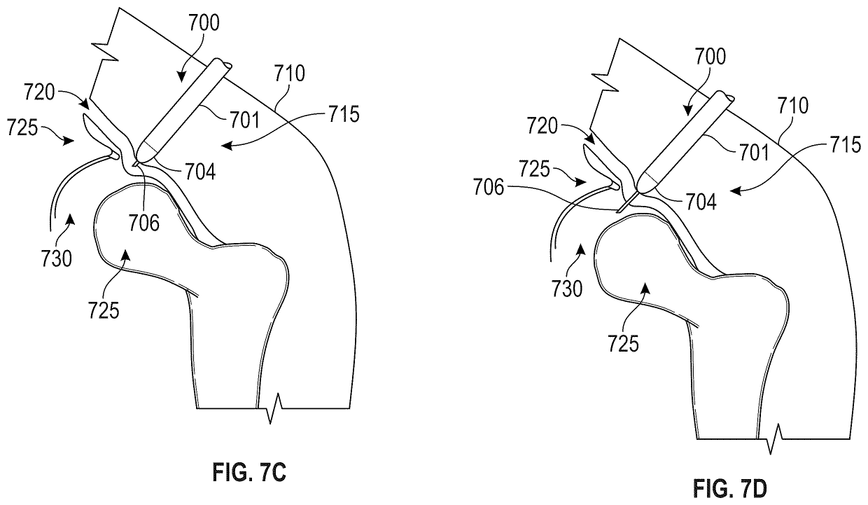

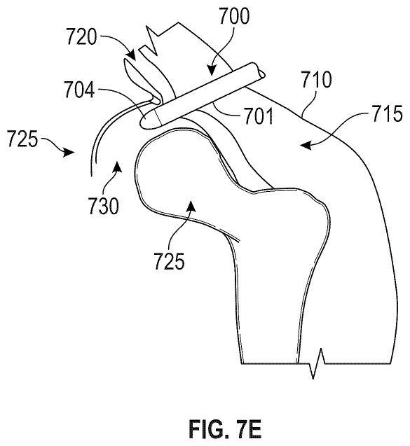

Although the hip, shoulder, and knee joints are discussed primarily throughout this description, it is to be understood that the palpating devices described herein can additionally or alternatively be used to palpate any synovial joint of a subject for optimized cannula placement. The joint capsule can be accessed by cutting a small incision through the skin and fascia of the subject proximate to the desired joint. The palpating device may be inserted through the incision and positioned proximate to the joint capsule. Various exemplary palpating devices are described herein with respect to FIGS. 1A-1C, 2, 3A-3C, 4A-4B, 5A-5D, and 6. In addition, exemplary methods for palpating and penetrating a joint capsule using the palpating devices provided herein are described with respect to FIGS. 7A-7E.

### Palpating Devices

FIGS. 1A-1C illustrate a palpating device 100 for palpating and penetrating a joint capsule of a subject, in accordance with aspects of the disclosure. The palpating device 100 includes a shaft 102 having a distal tip 104 configured for palpating a joint capsule of a subject to determine a suitable location for penetrating the joint capsule. The palpating device 100 includes a needle 106 disposed within the shaft 102 and configured for penetrating the joint capsule of the subject. The needle 106 may be similar to a spine needle in form and function. The palpating device 100 includes a handle 108 coupled to the shaft 102 and a slider 110 configured to translate the needle 106 between states of needle deployment, including a retracted state and one or more deployed states. The handle 108 may be fixed to the proximal end of the shaft 102 via one or more fasteners, adhesives, welds, or another means of attachment.

The slider 110 of the handle 108 can be coupled to the needle 106 so that moving the slider 110 causes translation of the needle 106. For example, a coupling portion 111 of the slider 110 disposed within an inner portion 109 of the handle 108 can be coupled to the needle 106. An engaging portion 113 of the slider 110 can be disposed at least partially external to the handle 108 so that the user can engage the slider 110 to move the needle 106. Each of the handle 108 and the slider 110 (e.g., at least the engaging portion 113 of the slider 110) may be ergonomically designed to allow the user to grasp the handle 108 and position the user's finger (e.g., thumb) on the engaging portion 113 of the slider 110. In this manner, the slider 110 can be moved by simply moving a finger.

The slider 110 may be configured to translate the needle 106 between a retracted state in which a distal end 124 of the needle 106 is retracted within the shaft 102 and a deployed state in which the distal end 124 of the needle 106 extends from the distal tip 104 of the shaft 102. With the needle 106 in the retracted state, the palpating device 100 can be used for palpating the joint capsule to identify a suitable location for penetrating the joint capsule. Once the suitable location is identified, the needle 106 can be translated to the deployed position for penetrating the joint capsule. Optionally, in one or both of the retracted state and the deployed state of the needle 106, the slider 110 may removably lock into place with the handle 108 so that the position of the needle 106 during use of the palpating device 100 is controlled.

In some examples, the slider 110 may be configured to translate the needle 106 between the retracted state and more than one deployed state, such as a partially deployed state for partially penetrating the joint capsule and a fully deployed state for fully penetrating the joint capsule. In the partially deployed state, the distal end 124 of the needle 106 can partially extend from the distal tip 104 of the shaft 102 to partially penetrate the joint capsule with the needle 106. In the fully deployed state, the distal end 124 of the needle 106 can fully extend from the distal tip 104 of the shaft 102 to fully penetrate the joint capsule with the needle 106. The user of the palpating device 100 may utilize the partially deployed state of the needle 106 to temporarily rest, or park, the needle 106 on the surface of the joint capsule and assess the position of the needle 106 (e.g., via fluoroscopic imaging) prior to fully penetrating the joint capsule. Additionally or alternatively, the partially deployed state of the needle 106 can be used to fully penetrate joint capsules having a thickness that is less than the length of the needle 106 partially extended from the distal tip 104 of the shaft 102. As described with respect to the deployed state generally, in one or both of the partially deployed state and the fully deployed state of the needle 106, the slider 110 may removably lock into place with the handle 108. The locking mechanism between the handle 108 and the slider 110 is described in greater detail below.

The handle 108 may include one or more locking positions 112a, 112b, and/or 112c configured to receive a corresponding locking member 114 protruding from the slider 110 to removably lock the slider 110 in place relative to the handle 108. The locking positions 112a, 112b, and/or 112c may include one or more indentations, or grooves, in a surface of the inner portion 109 within the handle 108. The locking member 114 may include a tab or other protrusion extending from a surface of the slider 110 that is sized to removably lock into each of the one or more locking positions 112a, 112b, and/or 112c. Each of the locking positions 112a, 112b, and 112c of the handle 108 may correspond to a state (e.g., retracted state, partially deployed state, and fully deployed state) of the needle 106 coupled to the slider 110. In FIG. 1A, the locking member 114 of the slider 110 is shown in the proximal-most locking position 112a. In this position, the needle 106 is in the retracted state the needle 106 is retracted within the shaft 102. In FIG. 1B, the locking member 114 of the slider 110 is shown in the intermediate locking position 112b. In this position, the needle 106 is in a partially deployed state—the distal end 124 of the needle 106 partially extends from the distal tip 104 of the shaft 102. In FIG. 1C, the locking member 114 of the slider 110 is shown in the distal-most locking position 112c. In this position, the needle 106 is in a fully deployed state the distal end 124 of the needle 106 fully extends from the distal tip 104 of the shaft 102.

The amount of movement of the slider 110 between each of the locking positions 112a, 112b, and 112c can correspond directly to the amount of movement of the needle 106 between each of the above-described states. For example, if the locking positions 112a and 112c were spaced apart by 30 mm, moving the slider 110 between the proximal-most locking position 112a and the distal-most locking position 112c on the handle 108 would cause the needle 106 to translate 30 mm.

A user of the palpating device 100 may move the slider 110 between the different locking positions 112a, 112b, and/or 112c by applying an inward force to the slider 110 that causes the locking member 114 to disengage from a given locking position 112a, 112b, or 112c. Applying an inward force to the slider 110 (i.e., toward the longitudinal axis 103 of the shaft 102) disengages locking member 114 from the given locking position 112a, 112b, or 112c. Specifically, the inward force applied to the slider 110 causes a living spring 116 of the slider 110—that is biased in a direction opposite the applied inward force—to bend toward the longitudinal axis 103 of the shaft 102, thereby disengaging the locking member 114 from the given locking position 112a, 112b, or 112c. Once the locking member 114 is disengaged from a given locking position 112a, 112b, or 112c, the user can apply force in the desired direction of movement of the slider 110 to translate the slider 110 and needle 106 coupled thereto. The slider 110 can translate along the longitudinal axis 103 of the shaft 102 and needle 106 disposed therein. The inner portion 109 of the handle 108 and the slider 110 may include corresponding surfaces that slidably engage, enabling the slider 110 to move along the longitudinal axis 103. The slider 110 may be translatable within a predetermined range of distances controlled by the proximal-most and distal-most locking positions 112a and 112c (e.g., corresponding to the retracted and fully deployed states). Additionally or alternatively, the amount of possible translation of slider 110 may be controlled by the geometry of the inner portion 109 within the handle 108 and the portion of the slider 110 disposed therein. Stated another way, the slider 110 may be translated in either direction until the slider 110 contacts a corresponding surface of the inner portion 109 of the handle 108. In this manner, the needle 106 coupled to the slider 110 may be retracted and/or extended beyond the states corresponding to the locking positions 112a, 112b, and/or 112c.

When the inward force applied to the slider 110 is released, the locking member 114 of the slider 110 may return to a locking position 112a, 112b, or 112c to removably lock the slider 110 back into place with the handle 108. As noted above, the living spring 116 of the slider 110 may be biased in the direction opposite to the applied force such that removing the applied force results in the living spring 116 returning the locking member 114 to a locking position 112a, 112b, or 112c. In some examples, the surface of the inner portion 109 within the handle 108 having the locking positions 112a, 112b, and/or 112c may be configured to guide and/or urge the locking member 114 toward one of the locking positions 112a, 112b, or 112c. For example, one or more portions of the surface may be angled and/or curved towards one or more of the locking positions 112a, 112b, and/or 112c. Thus, when the user releases the inward force applied to the slider 110 proximate to (but not precisely aligned with) one of the locking positions 112a, 112b, or 112c, the locking member 114 of the slider 110 moves into one of the locking positions 112a, 112b, or 112c without requiring the user to precisely align the locking member 114 with a given locking position 112a, 112b, or 112c.

In some examples, to retract the needle 106 back into the shaft 102, the user may engage with the slider 110 to manually retract the needle 106. Alternatively, in some examples, the needle 106 may be self-retractable, so that after the needle 106 is at least partially deployed from the distal tip 104 of the shaft 102, it may be configured to automatically retract into the shaft 102. For example, the needle 106 may be spring-loaded so that it can self-retract into the shaft 102 from a deployed state.

Although FIGS. 1A-1C illustrate three locking positions 112a, 112b, and 112c for removably locking the needle 106 in place, the palpating devices described herein are not intended to be limited to this arrangement. For example, an exemplary palpating device can include just two locking positions 112a and 112c (e.g., corresponding to a retracted state and a deployed state of a needle). In another example, an exemplary palpating device can include more than three (e.g., four or five) locking positions corresponding to additional deployed states of the needle 106. In some examples, the needle 106 may be extended to one or more states that do not correspond with a locking position 112a, 112b, or 112c. For example, the handle 108 may include only locking positions 112a and 112c, but the needle 106 may be extended to a partially deployed state between the two locking positions 112a and 112c (without locking into any locking position of the handle 108). In another example, as noted above, the needle 106 may be extended beyond the distance corresponding to the distal-most locking position 112c. In yet another example, the needle 106 may be retracted further within the shaft 102 than the distance corresponding to the proximal-most locking position 112a.

Moreover, although the locking mechanism of the palpating device 100 is described herein with reference to the locking member 114 protruding from the slider 110 and engaging with different locking positions 112a, 112b, and/or 112c on the handle 108, other examples can have the opposite configuration in which the slider 110 includes one or more locking positions (e.g., similar to locking positions 112a, 112b, and 112c) configured to correspond to a locking member (e.g., similar to locking member 114) and protruding from a surface of the handle 108.

As noted above, the distal tip 104 of the shaft 102 can be configured for palpating a joint capsule prior to penetrating the joint capsule. Thus, the distal tip 104 may be configured such that contacting the joint capsule with the distal tip 104 does not damage or penetrate the joint capsule, but instead provides the user of the palpating device 100 with a tactile response when the distal tip 104 contacts the joint capsule. The distal tip 104 of the shaft 102 may comprise a blunt distal-most end 118 oriented orthogonally to the longitudinal axis 103 of the shaft 102. For example, the blunt distal-most end 118 may be substantially flat or curved to facilitate palpating the joint capsule without penetrating the joint capsule. The distal tip 104 may have a conical or bullet-like shape that tapers to a minimum diameter at the blunt distal-most end 118. For example, a diameter of the shaft 102 (e.g., along a portion of the shaft 102 that is proximal to the distal tip 104) may be between 2-6 mm. A diameter of the blunt distal-most end 118 may be between 1-3 mm. This taper 120 of the distal tip 104 can facilitate insertion of the shaft 102 of the palpating device 100 through the joint capsule once the joint capsule is penetrated with the needle 106. The taper 120 of the distal tip 104 can dilate the joint capsule as the shaft 102 of the palpating device 100 is advanced through the joint capsule and into the joint space.

The shaft 102 can include a transition 122 in the region of the shaft 102 where the taper 120 begins. The diameter of the shaft 102 within the taper 120 can decrease toward a minimum diameter at the blunt distal-most end 118 of the distal tip 104 of the shaft 102. The transition 122 may be a continuous curve to the distal tip 104 of the shaft 102 to facilitate inserting the distal portion of the shaft 102 through the joint capsule. In this manner, the shaft 102 can be smoothly inserted through the joint capsule following dilation from the taper 120 of the distal tip 104 without the transition 122 catching on the joint capsule and potentially causing tissue damage to the subject. In some examples, the transition 122 may be set back from the blunt distal-most end 118 of the distal tip 104 to facilitate smooth dilation and entry of the distal tip 104 of the shaft 102 through the joint capsule. For example, the transition 122 may be set back 3-8 mm from the blunt distal-most end 118 of the distal tip 104. In some examples, the distal tip 104, including the transition 122, may have a smooth surface or include textures. For example, the surface of distal tip 104 may include fine threads and/or ribs disposed about the distal tip 104 to further facilitate penetrating the joint capsule.

In some examples, the shaft 102 of the palpating device 100 may be compatible with one or more dilators (not illustrated) for dilating the joint capsule. The dilator(s) may include a lumen and the shaft 102 may be sized to be slidably received in the dilator(s) to dilate the joint capsule for a cannula.

The distal end 124 of the needle 106 may be angled relative to the longitudinal axis 103 of the needle 106 to provide a sharp point for penetrating the joint capsule. The angle of the distal end 124 of the needle 106 may form a leading tip 126 that initially contacts and penetrates the joint capsule. The orientation of the leading tip 126 of the needle 106 relative to the anatomy of the joint may be important to consider when penetrating the joint capsule to prevent unintentionally poking, penetrating, or otherwise causing damage to tissues or bony structures in the joint space. Thus, to enhance the user's awareness of the orientation of the leading tip 126 of the needle 106, the leading tip 126 may be aligned with one or more other features of the palpating device 100. For example, as shown in FIGS. 1A-1C, the leading tip 126 is aligned with the side of the handle 108 that includes the slider 110. In this manner, the leading tip 126 of the needle 106 can be easily oriented during use of the palpating device 100. Understanding the orientation of the leading tip 126 of the needle 106 relative to other features of the palpating device 100 can also aid in understanding the overall orientation of the palpating device 100 relative to the target site of the subject. For example, during imaging of the joint, the user may visualize the leading tip 126 of the needle 106 in the joint capsule and/or joint space of the subject and may use the orientation of the leading tip 126 to indicate overall orientation of the palpating device 100 in space.

Although FIGS. 1A-1C illustrate an example in which the leading tip 126 of the needle 106 is oriented with the side of the handle 108 including the slider 110, it is to be understood that the leading tip 126 may have any orientation. For example, the leading tip 126 may be oriented with the side of the handle 108 opposite the side that includes the slider 110. Optionally, one or more components of the palpating device 100 (e.g., the handle 108 and/or the shaft 102) that are external to the subject during the use of the palpating device 100 may include a visual indication or other marking that indicates the orientation of the leading tip 126 of the needle 106.

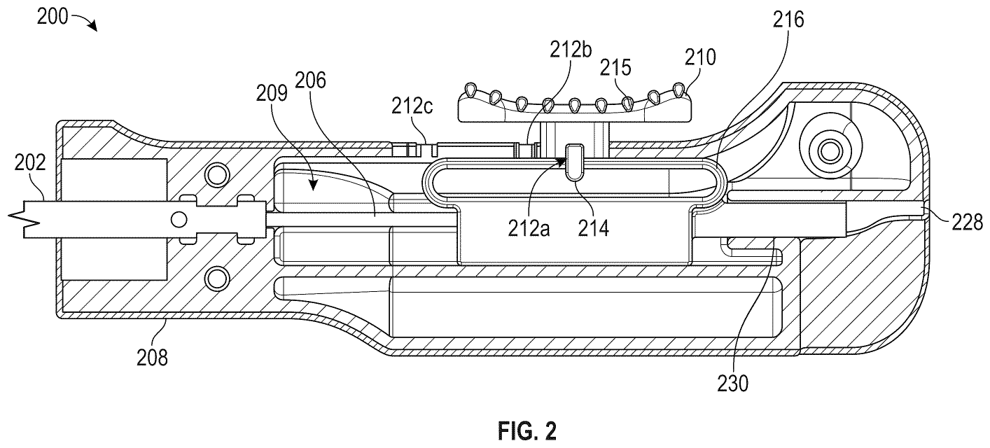

FIG. 2 illustrates a cross-sectional view of another configuration of a palpating device. Palpating device 200 of FIG. 2 includes a handle 208 and a slider 210 that are similar in function to the handle 108 and slider 110 of palpating device 100 of FIGS. 1A-1C. Each of handle 208 and slider 210, like handle 108 and slider 110, may be ergonomically designed to enable a user of the palpating device 200 to easily grip the handle 208 and engage the slider 210 with just one finger (e.g., a thumb). An outer surface 215 of the slider 210 may provide traction for the user when engaging with the slider 210 to move it. For example, the outer surface 215 can include one or more bumps, grooves, etchings, etc. that create traction between the user's finger and the slider 210. The slider 210 may be curved inward toward the handle 208 to accommodate the possible curvature of the finger that engages the slider 210.

The slider 210 can include a living spring 216 disposed within an inner portion 209 of the handle 208. The living spring 216 may be annular or ovular in shape. When the user applies an inward force to the slider 210 (e.g., to the outer surface 215 of the slider 210) to move the slider 210, the living spring 216 may bend to disengage the locking member 214 from a locking position 212a, 212b, or 212c of the handle 208. When the user releases the inward force applied to the slider, the living spring 216 may return the locking member 214 to a locking position 212a, 212b, or 212c of the handle 208. As described above with respect to the palpating device 100 depicted in FIGS. 1A-1C, the user may engage with the slider 210 to retract and extend the needle 206 coupled to the slider 210. When force in the desired direction of movement of the slider 210 is applied to the slider 210 and the locking member 214 is displaced from a given locking position (e.g., 212a), the slider 210 (and the needle 206 coupled thereto) can be moved to a different locking position (e.g., 212b or 212c).

The slider 210 may include a guidewire receiving portion 230 at a proximal end of the portion of the slider 210 disposed within the inner portion 209 of the handle 208. The guidewire receiving portion 230 can be configured to receive a guidewire through the guidewire lumen 228 of the handle 208 and can feed the guidewire into the guidewire lumen of the slider 210. The proximal end of the guidewire receiving portion 230 may contact the guidewire lumen 228 of the handle 208 to prevent the guidewire from entering the inner portion 209 of the handle 208 as it is being passed into the guidewire lumen of the slider 210. In some examples, the proximal end of the guidewire receiving portion 230 may contact the guidewire lumen 228 when the locking member 214 is in the proximal-most locking position 212a (as illustrated in FIG. 2). In some examples, the guidewire receiving portion 230 may additionally prevent translation of the slider 210 in a proximal direction beyond the proximal-most locking position 212a.

In a similar nature, in some examples, a distal-most portion of the slider 210 (e.g., the distal portion of the living spring 216 in the example illustrated in FIG. 2) may be configured to contact a distal end of the inner portion 209 of the handle 208 to prevent translation of the slider 210 in a distal direction beyond the distal-most locking position 212c.

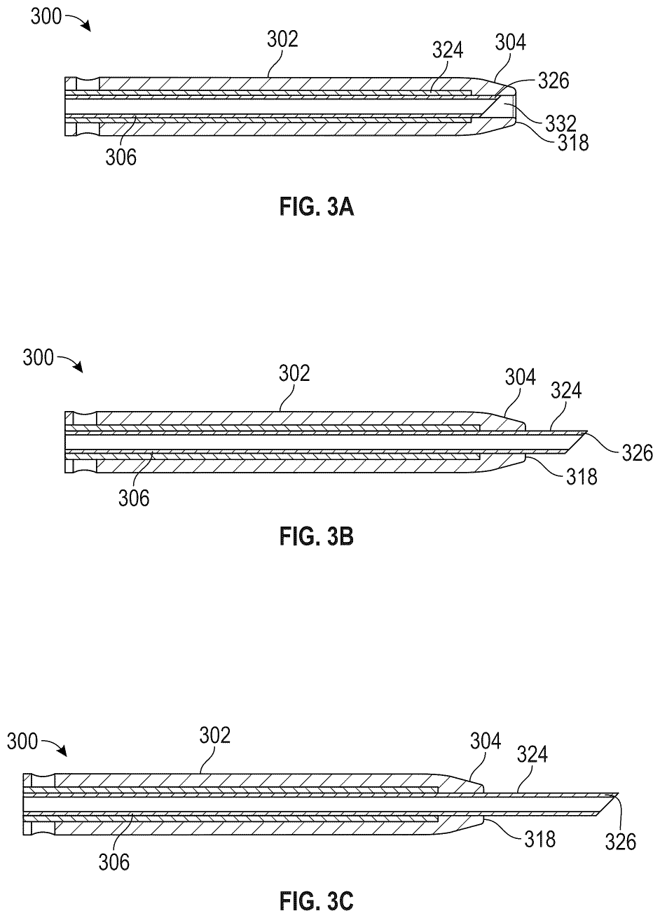

As noted above, movement of a slider (e.g., slider 110 of FIGS. 1A-1C or slider 210 of FIG. 2) relative to a handle (e.g., handle 108 of FIGS. 1A-1C or handle 208 of FIG. 2) of the palpating device causes a needle (e.g., needle 106 of FIGS. 1A-1C or needle 206 of FIG. 2) disposed within a shaft (e.g., shaft 102 of FIGS. 1A-1C or shaft 202 of FIG. 2) to retract into and extend from the distal tip of the shaft for palpating and penetrating a joint capsule. FIGS. 3A-3C are cross-sectional views of an exemplary distal end 300 of a palpating device (such as palpating device 100 or palpating device 200 of FIG. 2) illustrating states of a needle 306 disposed within a lumen 332 of a shaft 302. The shaft 302 and needle 306 are representative of the shaft and needle of any of the palpating devices described herein (e.g., palpating device 100 and 200, as well as palpating devices 500 and 600 described in greater detail below).

FIG. 3A illustrates the needle 306 in a retracted state in which the distal end 324 of the needle 306 is retracted within the shaft 302. As noted above, in the retracted state, the distal tip 304 of the shaft 302 can be used for palpating the joint capsule. In the retracted state, the distal end 324 of the needle 306 can be retracted at least 1 mm from the distal tip 304 of the shaft 302. Specifically, the distance by which the distal end 324 of the needle 306 is retracted into the shaft 302 may be measured between a blunt distal-most end 318 of the distal tip 304 of the shaft 302 and a leading tip 326 of the distal end 324 of the needle 306. In some examples, the distal end 324 of the needle 306 can be retracted between about 0-5 mm, 0.5-3 mm, or 1-2 mm from the distal tip 304 of the shaft 302. In some examples, the distal end 324 of the needle 306 can be retracted to at least 0, 0.2, 0.5, 0.8, 1, 1.5, 2, 2.5, 3, 3.5, 4, or 4.5 mm from the distal-most end 318 of the distal tip 304 of the shaft 302. In some examples, the distal end 324 of the needle 306 can be retracted to at least 0.2, 0.5, 0.8, 1, 1.5, 2, 2.5, 3, 3.5, 4, 4.5, or 5 mm from the distal-most end 318 of the distal tip 304 of the shaft 302.

FIG. 3B illustrates the needle 306 in the partially deployed state in which the distal end 324 of the needle 306 partially extends from the distal tip 304 of the shaft 302. As noted above, in the partially deployed state, the needle 306 may partially penetrate the joint capsule, for example, to assess the position of the needle 306 in the joint capsule prior to fully penetrating the joint capsule. In some examples, in the partially deployed state, the user may image the joint with the palpating device parked thereon to confirm the position of the needle 306 on the joint capsule. In some examples, the partially deployed state of the needle 306 may additionally or alternatively be effective in fully penetrating a joint capsule, for example, for instances in which the thickness of the joint capsule is less than the length of the needle 306 extended from the distal tip 304 of the shaft 302. In the partially deployed state, the distal end 324 of the needle 306 may extend between greater than 0 mm to 10 mm from the distal tip 304 of the shaft 302. The distance by which the distal end 324 of the needle 306 extends from the shaft 302 may be measured between the blunt distal-most end 318 of the distal tip 304 of the shaft 302 and the leading tip 326 of the distal end 324 of the needle 306. In some examples, the distal end 324 of the needle 306 can extend between about 1-8, 2-7, or 4-6 mm from the distal tip 304 of the shaft 302 in the partially deployed state. In some examples, the distal end 324 of the needle 306 can extend to a length of 0, 1, 2, 3, 4, 5, 6, 7, 8, or 9 mm from the distal-most end 318 of the distal tip 304 of the shaft 302 in the partially deployed state. In some examples, the distal end 324 of the needle 306 can extend to a length of 1, 2, 3, 4, 5, 6, 7, 8, 9, or 10 mm from the distal-most end 318 of the distal tip 304 of the shaft 302 in the partially deployed state. In these examples, there may be none, one, or multiple partially deployed states of the needle 306.

FIG. 3C illustrates the needle 306 in the fully deployed state relative to the shaft 302. As noted above, in the fully deployed state, the needle 306 can fully penetrate the joint capsule. In the fully deployed state, the distal end 324 of the needle 306 extends between a length that is greater than 10 mm to 30 mm from the distal tip 304 of the shaft 302. In some examples, the distal end 324 of the needle 306 can extend between a length that is about 12-28, 15-25, or 18-22 mm from the distal tip 304 of the shaft 302 in the fully deployed state. In some examples, the distal end 324 of the needle 306 can extend to a length of 10, 12, 15, 18, 20, 22, 25, or 28 mm from the distal-most end 318 of the distal tip 304 of the shaft 302 in the fully deployed state. In some examples, the distal end 324 of the needle 306 can extend to a length of 12, 15, 18, 20, 22, 25, 28, or 30 mm from the distal-most end 318 of the distal tip 304 of the shaft 302 in the fully deployed state.

The thickness of a typical joint capsule can vary between about 5-30 mm depending on the size (e.g., weight/height), age, etc. of the subject. Accordingly, the palpating devices described herein may account for the various possible thicknesses of the joint capsule by providing a needle 306 that can be deployed from the palpating device (e.g., palpating device 100) to penetrate both thinner joint capsules (e.g., joint capsules having a thickness around 5 mm) and thicker joint capsules (e.g., joint capsules having a thickness around 30 mm). In some examples, the palpating devices described herein may account for the various angles of entry in which a user may introduce the palpating device into a subject to reach the joint. The more orthogonally the palpating device approaches the joint capsule, the less amount of joint capsule tissue the needle will need to penetrate to enter the joint space.

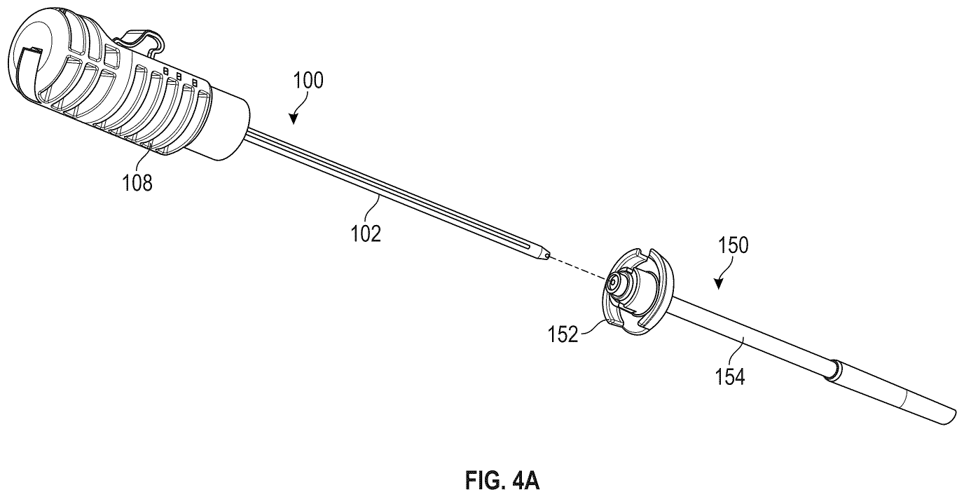

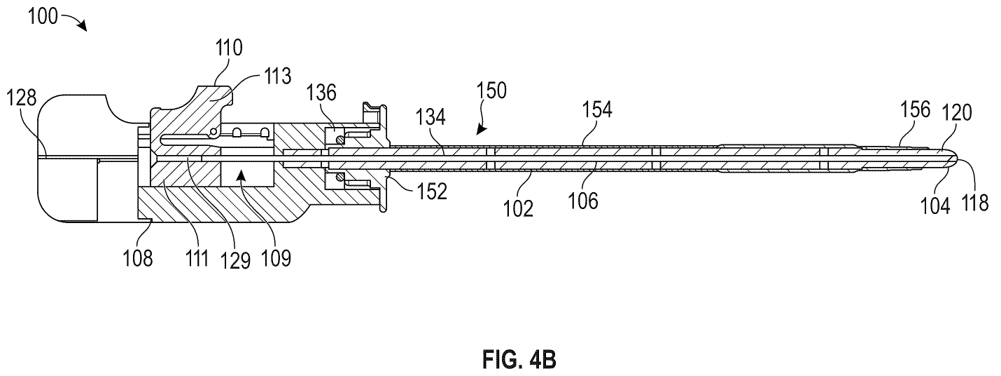

As noted above, the palpating devices described herein may be used to create a path through a joint capsule and into a joint space surrounding a target joint for further procedures on the joint. Accordingly, the palpating devices described herein can include, or be configured to be removably attached to, a cannula. The cannula can be used to establish a portal into the joint space in the path through the joint capsule created by the palpating device. FIG. 4A depicts an exploded view of the palpating device 100 and a cannula 150 configured to be removably attached to the palpating device 100. The cannula 150 can include a connection portion 152 and a shaft 154. The handle 108 may removably attach to the connection portion 152 when the shaft 102 of the palpating device 100 is received by the shaft 154 of the cannula 150. The shaft 102 of the palpating device 100 may be received by a lumen of the shaft 154 so that the shaft 102 of the palpating device 100 and the shaft 154 of the cannula 150 are slidably engaged. The palpating device 100 and cannula 150 may be assembled prior to use of the palpating device 100.

The shaft 102 of the palpating device 100 may be sized to be received by the cannula 150. The cannula 150 can vary in size from between about 2.5 mm to 6.5 mm in inner diameter (i.e., diameter of the lumen within the cannula 150). Accordingly, the shaft 102 may be appropriately sized to be received by the selected cannula 150. For example, the shaft 102 may have an outer diameter in a range of about 2-6.5 mm, 2.5-6 mm, 3-5.5 mm, or 3.5-5 mm. In some examples, the outer diameter of the shaft 102 may be at most about 2.5, 3, 3.5, 4, 4.5, 5, 5.5, 6, or 6.5 mm. In some examples, the outer diameter of the shaft 102 may be at least about 2, 2.5, 3, 3.5, 4, 4.5, 5, 5.5, or 6 mm.

FIG. 4B depicts the connection between the palpating device 100 and the cannula 150, with the shaft 102 of the palpating device 100 received by the cannula 150. The handle 108 includes a receiving portion 136 at the distal end of the handle 108 that is configured to receive the connection portion 152 of the cannula 150. Each of the receiving portion 136 of the handle 108 and the connection portion 152 of the cannula 150 may have corresponding mating geometries to enable the palpating device 100 and the cannula 150 to be removably attached. For example, the receiving portion 136 and the connection portion 152 may removably attach via a snap fit, press fit, threaded attachment, or another means of attachment.

When the shaft 102 of the palpating device 100 is received in the shaft 154 of the cannula 150, at least the distal tip 104 of the shaft 102 may protrude from a distal-most end 156 of the shaft 154 of the cannula 150 to ensure the palpating device 100 can dilate the opening created by the needle 106 prior to inserting the cannula 150 attached thereto (e.g., at least in part due to the taper 120 of the distal tip 104). For example, at least 4-8 mm of the distal portion of the shaft 102 (including the distal tip 104) may protrude from the distal-most end 156 of the shaft 154. The amount that the distal portion of the shaft 102 protrudes from the shaft 154 may be measured between the blunt distal-most end 118 of the shaft 102 of the palpating device 100 and the distal-most end 156 of the shaft 154 of the cannula 150. In some examples, the distal-most end 156 of the cannula 150 may have an outer diameter similar to that of the shaft 102 so that the palpating device 100 and cannula 150 attached thereto can be smoothly inserted through the joint capsule. The cannula 150 may taper in diameter toward the distal-most end 156 of the shaft 154 to continue to dilate the opening created in the joint capsule as the shaft 102 of the palpating device 100 and the shaft 154 of the cannula 150 attached thereto are advanced into the joint space.

The cross-sectional view in FIG. 4B also shows that each of the needle 106, the handle 108, and the slider 110 include a guidewire lumen for passing a guidewire through the palpating device 100 and into the joint space after the needle 106 has penetrated the joint capsule. The guidewire lumen 128 of the handle 108 can extend from a proximal end of the handle 108 into the inner portion 109 of the handle 108. The guidewire lumen 128 can be operably connected with the guidewire lumen 129 extending within the coupling portion 111 of the slider 110. The guidewire lumen 129 of the slider 110 can be operably connected with the guidewire lumen 134 extending within the needle 106. Thus, a guidewire can be fed through the guidewire lumen 128 of the handle 108, through the guidewire lumen 129 of the slider 110, through the guidewire lumen 134 of the needle 106, and out of the distal end 124 of the needle 106 for deployment in the joint space. After the guidewire is in place in the joint space, the needle 106 can be withdrawn back into the shaft 102 to avoid damage to the tissue of the joint capsule.

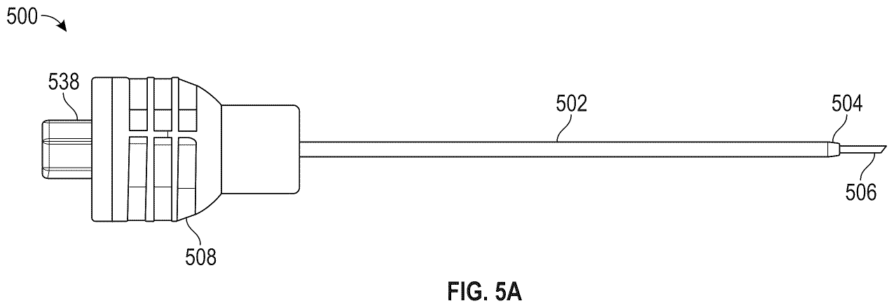

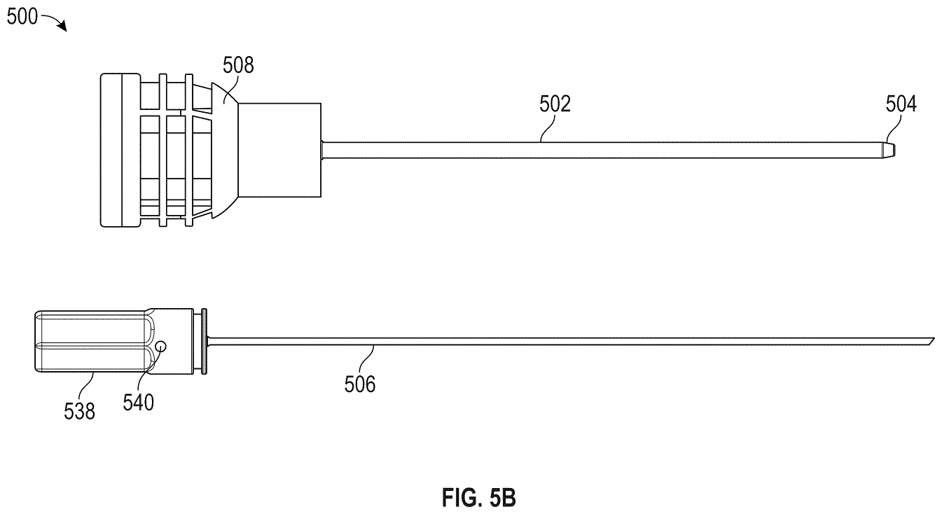

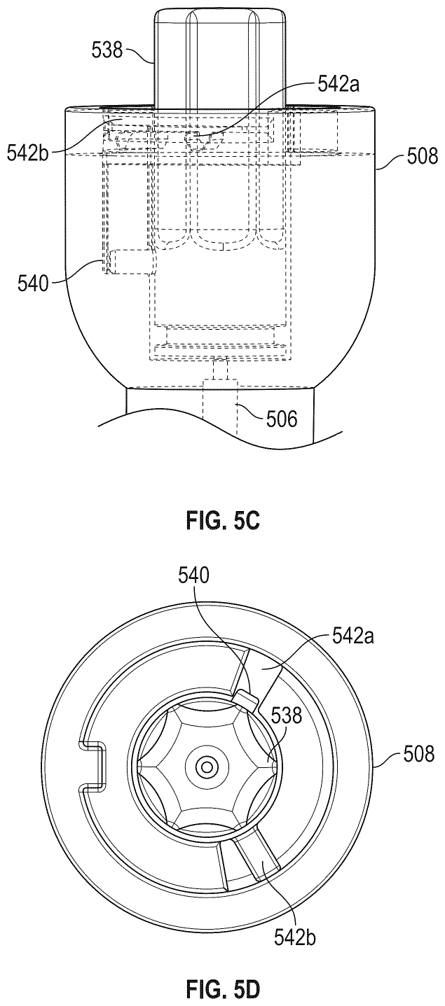

FIGS. 5A-5D illustrate another example of a palpating device for palpating and penetrating a joint capsule. The palpating device 500 of FIGS. 5A-5D includes a needle 506 that may be decoupled from the handle 508. FIG. 5A depicts the needle 506 assembled with the remainder of the palpating device 500, whereby the needle 506 is disposed within the shaft 502 and handle 508 of the palpating device 500. FIG. 5B depicts the needle 506 disassembled from the remainder of the palpating device 500.

The needle 506 may be inserted into the shaft 502 of the palpating device 500 so that a proximal portion 538 of the needle 506 engages with an inner portion of the handle 508. Shown in FIGS. 5C-5D, the proximal portion 538 of the needle 506 can include a locking member 540 that engages with locking positions 542a and 542b within the handle 508. The locking member 540 may be a pin or other protrusion extending from the main body of the proximal portion 538 of the needle 506. The locking positions 542a and 542b may be grooves or tracks within the inner portion of the handle 508 that are sized to receive the locking member 540. The position of the locking member 540 of the proximal portion 538 of the needle 506 relative to the locking positions 542a and 542b within the handle 508 can control the distance by which the distal end of the needle 506 extends from the distal tip 504 of the shaft 502. For example, when the needle 506 is initially inserted through the handle 508 and into the shaft 502 of the palpating device 500, the needle 506 may be advanced until the locking member 540 of the proximal portion 538 of the needle 506 engages the first locking position 542a. FIG. 5D illustrates the locking member 540 removably locked into the first locking position 542a. In the first locking position 542a, the needle 506 may be in a retracted state, whereby the needle 506 is retracted within the shaft 502 of the palpating device 500 for palpating a joint capsule with the distal tip 504 of the shaft 502.

To further advance the needle 506 through the shaft 502 of the palpating device 500 so that the needle 506 extends from the distal tip 504 of the shaft 502, the proximal portion 538 of the needle 506 may be rotated within the handle 508. The first locking position 542a may be connected to the second locking position 542b by a track within the inner portion of the handle 508, so that rotating the proximal portion 538 of the needle 506 guides the locking member 540 of the proximal portion 538 along the track and to the second locking position 542b. FIG. 5C illustrates the locking member 540 removably locked into the second locking position 542b. In the second locking position 542b, the needle 506 may be in a deployed state, whereby the needle 506 is extended from the distal tip 504 of the shaft 502 for penetrating a joint capsule with the needle 506.

Although only two locking positions 542a and 542b in the handle 508 are described with respect to FIGS. 5C-5D, it is to be understood that the handle 508 may include more than two (e.g., three, four, or more) locking positions that correspond with different states of the needle 506 relative to the shaft 502. For example, the handle 508 of palpating device 500 may include at least three locking positions, as described with respect to the palpating devices 100 and 200 illustrated in FIGS. 1A-1C and FIG. 2, that correspond to a retracted state, partially deployed state, and a fully deployed state of the needle 506 from the shaft 502. In other examples, the handle 508 of palpating device 500 may include any number of locking positions suitable for facilitating different states of the needle 506 relative to the shaft 502.

The needle 506 may be used either with or without the remainder of the palpating device 500. For example, the needle 506 may be used like a spine needle to penetrate tissue (e.g., a joint capsule) without palpating the tissue with the palpating device 500. When the needle 506 is inserted into the shaft 502 of the palpating device 500 and retracted within the shaft 502, the distal tip 504 of the shaft 502 may be used to palpate the tissue prior to penetrating the tissue with the needle 506 when the needle 506 is extended from the distal tip 504 of the shaft 502.

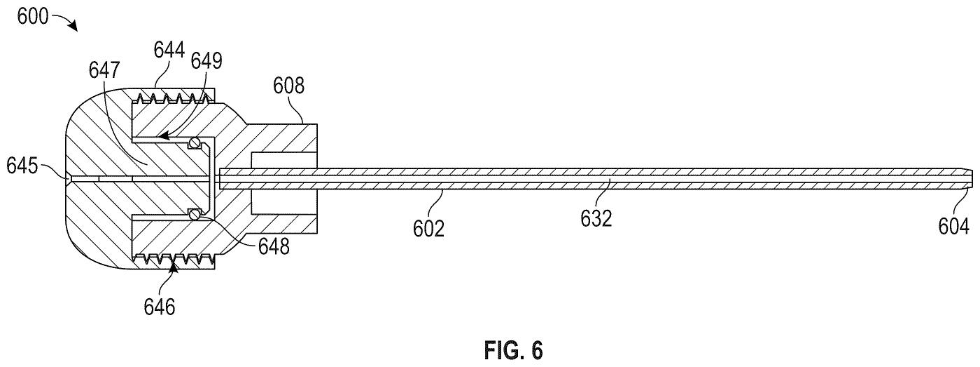

FIG. 6 illustrates another example of a palpating device for palpating and penetrating a joint capsule. The palpating device 600 of FIG. 6 includes a threaded mechanism for translating a needle (not illustrated in FIG. 6) through the shaft 602 of the palpating device 600. The threaded mechanism can include a rotator 644 for translating a needle (not shown) that is coupled to the rotator 644. The rotator 644 may be coupled to the handle 608 via a threaded portion 646 fitting 648, so that rotating the rotator 644 relative to the handle 608 causes the needle to translate through the shaft 602, and the rotator 644 to translate relative to the handle 608. The threaded portion 646 may include threads on an inner surface of the rotator 644 and corresponding threads on an outer surface of the handle 608. The fitting 648 may be disposed at least partially within an inner portion 647 of the rotator 644, between the inner portion 647 of the rotator 644 and the inner portion 649 of the handle 608. Using the palpating device 600, a needle coupled to the rotator 644 and disposed within the lumen 632 of the shaft 602 may be translated continuously from a retracted state within the shaft 602 for palpating a joint capsule to a deployed state external from the distal tip 604 of the shaft 602 for penetrating the joint capsule by rotating the rotator 644. The palpating device 600 illustrated FIG. 6 also includes a guidewire lumen 645 that is configured to receive a guidewire.

### Methods for Palpating & Penetrating a Joint Capsule

FIGS. 7A-7E illustrate aspects of a method for palpating and penetrating a joint capsule surrounding a joint. The palpating device 700 depicted in FIGS. 7A-7E is understood to be representative of any of the palpating devices 100, 200, 500, and/or 600 described herein. Furthermore, although not explicitly illustrated, it is to be understood that the palpating and penetrating methods depicted in FIGS. 7A-7E are applicable to a palpating device having a cannula removably attached thereto (e.g., palpating device 100 and cannula 150, illustrated in FIGS. 4A-4B). The methods described herein with respect to FIGS. 7A-7E are understood to be applicable to palpating and penetrating joint capsules surrounding various types of joints, including hips, knees, shoulders, and other synovial joints.

Palpating and penetrating a joint capsule may include cutting a small incision in the skin 710 and tissue (e.g., fascia) proximate to a target site, including a joint of interest. A distal portion 701 of the palpating device 700 can be inserted through the incision and into the target site 715 to contact the joint capsule 720. As necessary, the palpating device 700 may be maneuvered around any obstructing anatomical structures to contact the joint capsule 720 with the distal portion 701 of the palpating device 700. Once the palpating device 700 is in contact with the joint capsule 720, the joint capsule 720 can be palpated using a distal tip 704 of the palpating device 700 to determine an appropriate, or compliant, location for penetrating the joint capsule 720. Palpating the joint capsule 720 can include the user pressing and repositioning the distal portion 701 of the palpating device 700 to determine the underlying anatomical structures (e.g., bony anatomy 725). The user can press and reposition the palpating device interchangeably and/or at the same time (i.e., move the distal portion 701 of the palpating device 700 while applying pressure to the joint capsule 720). In FIG. 7A, the palpating device 700 may be positioned at a location of the joint capsule 720 overlying bony anatomy 725. As the user palpates in this position, the user may recognize that the location is relatively uncompliant, which can indicate an inappropriate position for penetrating the joint capsule 720 due to the nearby bony anatomy 725. The user may reposition the palpating device 700 and continue to palpate to locate a section of the joint capsule 720 that is more compliant (e.g., the same amount of palpating force results in greater movement of the palpating device 700). In FIG. 7B, the palpating device 700 is pressed into a portion of the joint capsule that is overlying the joint space 730. The user may perceive that this location of the joint capsule 720 is more compliant, which can indicate to the user that the palpating device 700 is located at a distracted space in the joint. The user may determine that this location is appropriate for creating a portal to the joint.

Upon determination of the appropriate location for penetrating the joint capsule 720, a needle 706 of the palpating device 700 can be extended to penetrate the joint capsule 720. In some examples, as shown in FIG. 7C, the joint capsule 720 can be partially penetrated prior to fully penetrating the joint capsule 720. This may be useful to assess whether the position of the needle 706 in the joint capsule 720 is appropriate for fully penetrating the joint capsule 720. For example, the user may confirm whether the palpating device 700 is in the appropriate position for penetrating the joint capsule 720 by taking fluoroscopic images of the target site 715 while the palpating device 700 is inserted therein, with the needle 706 parked within the joint capsule 720. Once the user confirms that the position of the palpating device 700 is appropriate, the needle 706 can be fully deployed to fully penetrate the joint capsule 720, as illustrated in FIG. 7D. In some examples, the needle 706 may be partially deployed to fully penetrate the joint capsule 720, especially in areas where the joint capsule 720 is relatively thin.

Optionally, after penetrating the joint capsule 720, a guidewire can be inserted through a lumen of the palpating device 700 and through the joint capsule 720. For example, the palpating device 700 (e.g., at least the needle 706 of the palpating device 700) can include a lumen through which a guidewire can be inserted to be deployed in the joint space 730. The guidewire can guide placement of a cannula through the joint capsule 720 and into the joint space 730.

After penetrating the joint capsule 720, the needle 706 can be retracted from the joint capsule 720. In instances in which a guidewire is inserted through the palpating device 700 and into the joint space 730, the needle 706 may be retracted before or after the guidewire is positioned in the joint space 730.

After penetrating the joint capsule 720, the distal portion 701 of the palpating device 700 can be inserted through the joint capsule 720 and into the joint space 730, as shown in FIG. 7E. The distal portion 701 of the palpating device 700 may be inserted through the joint capsule 720 either before or after the needle 706 is retracted; however, it may be preferable to insert the distal portion 701 of the palpating device 700 after retracting the needle 706 to avoid accidentally penetrating any tissue in the joint space 730. In instances in which the palpating device 700 is used with a guidewire, the distal portion 701 of the palpating device 700 may be inserted through the joint capsule 720 either before or after the guidewire is passed through the palpating device 700.

As noted above, the method illustrated in FIGS. 7A-7E may be executable with a palpating device having a cannula attached thereto. Accordingly, in these instances, once the path through the joint capsule 720 has been created by the palpating device, the cannula attached to the palpating device may be inserted through the joint capsule 720. Once a portion of the cannula is passed through the joint capsule 720 and into the joint space 730, the palpating device may be removed from within the cannula, and the cannula may be left in place in the joint capsule 720 to establish a portal through the joint capsule 720 and into the space surrounding the joint. Through this portal, additional procedures on the joint can proceed.

Inserting a cannula through the joint capsule 720 may take place either with or without the use of a guidewire. In aspects in which the cannula is attached to the palpating device as the palpating device penetrates and is inserted through the joint capsule 720, the guidewire may be optional. Alternatively, the palpating device 700 (e.g., without the cannula attached thereto) may be used to palpate and penetrate the joint capsule 720, and once penetrated, a guidewire can be passed through the palpating device 700 and into the joint space 730. The palpating device 700 and cannula may then be inserted through the joint capsule 720 along the guidewire. After the cannula is appropriately placed, the guidewire and palpation device can be removed from the target site 715.

As noted above, the palpating devices described herein may optionally be used with dilators to dilate the joint capsule after it is penetrated by the needle. Accordingly, increasingly larger dilators (or cannulae) that are removably attached to the shaft of the palpating device can be inserted into the path created in the joint capsule 720 until the path reaches a desired size.

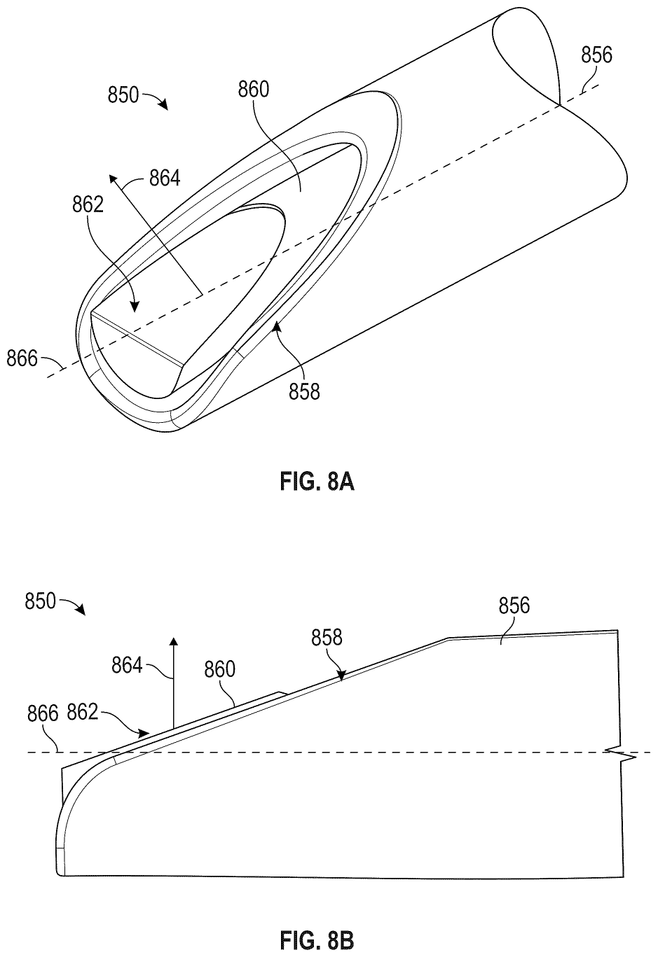

As described herein, once a cannula is properly placed in the joint space, the palpating device can be removed from the cannula, and the cannula can be left in place in the joint space. A n endoscope can then be inserted into the joint space via the cannula. Different configurations of the cannula may be used for different types of endoscopes. For example, a cannula configured for receiving endoscopes that have angled distal ends may have an angled distal end that corresponds to the angled distal end of the endoscope. FIGS. 8A-8B illustrate a distal portion of an exemplary cannula 850 having an angled distal end that corresponds to an angled distal end of an exemplary endoscope 860 disposed therein. Cannula 850 is understood to be representative of any of the cannulae described herein (e.g., cannula 150 of FIG. 4A).

Endoscope 860 includes an angled distal end 862 that provides a viewing direction 864 that is transverse to a longitudinal axis 866 of the endoscope 860. Endoscope 860 is merely exemplary and is representative of any endoscope having an angled distal end.

The distal portion of the shaft 856 of the cannula 850 can include a cutout that results in an angled distal end 858 corresponding to the angled distal end 862 of the endoscope 860. In this way, endoscope 860 can provide the field of view without requiring the angled distal end 862 of the endoscope 860 to be advanced distally beyond the distal end of the cannula 850. This arrangement may be advantageous, for example, in scenarios where the space within the surgical cavity (e.g., the joint space) is limited. In some aspects, the angle of the angled distal end 858 of the cannula 850 can be substantially the same as the angle of the angled distal end 862 of the endoscope 860.

As shown in FIG. 8B, the angled distal end 862 of the endoscope 860 may stand proud of the angled distal end 858 of the cannula 850 when the distal end of the endoscope 860 is substantially aligned with the distal end of the cannula 850. In some examples, the angled distal end 862 of the endoscope 860 stands proud of the angled distal end 858 of the cannula 850 by about a millimeter or less. In this way, the field of view of the camera within the endoscope 860 can remain clear of the cannula 850.

In some aspects, the angled distal end 862 of the endoscope 860 is flush with the angled distal end 858 of the cannula 850. In some aspects, the angled distal end 862 of the endoscope 860 is recessed from the angled distal end 858 of the cannula 850. For example, the angled distal end 862 of the endoscope 860 may be recessed from the angled distal end 858 of the cannula 850 by about a millimeter or less.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

For the purpose of clarity and a concise description, features are described herein as part of the same or separate examples; however, it will be appreciated that the scope of the disclosure includes examples having combinations of all or some of the features described.

What is claimed is:

1. A device for palpating and penetrating a joint capsule of a subject, comprising:
a shaft having a distal tip configured for palpating the joint capsule of the subject;
a needle disposed within the shaft and configured to penetrate the joint capsule of the subject; and
a handle coupled to the shaft and comprising a slider configured to translate the needle between a retracted state in which a distal end of the needle is retracted within the shaft and a fully deployed state in which the distal end of the needle fully extends from the distal tip of the shaft.

2. The device of claim 1, wherein the slider removably locks into place with the handle when the needle is in the retracted state and the fully deployed state.

3. The device of claim 1, wherein the distal end of the needle is retracted at least 1 mm from the distal tip of the shaft in the retracted state.

4. The device of claim 1, wherein the distal end of the needle extends between a length that is greater than 10 mm to 30 mm from the distal tip of the shaft in the fully deployed state.

5. The device of claim 1, wherein the slider is configured to translate the needle between the retracted state and a partially deployed state in which the distal end of the needle partially extends from the distal tip of the shaft.

6. The device of claim 5, wherein the slider removably locks into place with the handle when the needle is in the partially deployed state.

7. The device of claim 5, wherein the distal end of the needle extends between a length that is greater than 0 mm to 10 mm from the distal tip of the shaft in the partially deployed state.

8. The device of claim 1, wherein each of the needle, the handle, and the slider comprises a guidewire lumen.

9. The device of claim 1, wherein the handle is configured to removably attach to a cannula configured to receive the shaft.

10. The device of claim 1, wherein a leading tip of the needle is aligned with a side of the handle comprising the slider for orienting the leading tip of the needle.

11. The device of claim 1, wherein the distal tip comprises a blunt distal-most end oriented orthogonally to a longitudinal axis of the shaft.

12. The device of claim 11, wherein a diameter of the distal tip of the shaft tapers to a minimum diameter at the blunt distal-most end to facilitate inserting a distal portion of the shaft through the joint capsule.

13. The device of claim 1, wherein the shaft comprises a continuous curve to the distal tip of the shaft to facilitate inserting a distal portion of the shaft through the joint capsule.

14. A method for palpating and penetrating a joint capsule of a subject, comprising:
inserting a distal portion of a palpating device into a target site to contact the joint capsule of the subject;
palpating the joint capsule using a distal tip of the palpating device to determine an appropriate position for penetrating the joint capsule; and
upon determination of the appropriate position, extending a needle of the palpating device to penetrate the joint capsule.

15. The method of claim 14, comprising, prior to fully extending the needle of the palpating device to penetrate the joint capsule, imaging the target site having the palpating device inserted therein to determine whether the palpating device is in the appropriate position for penetrating the joint capsule.

16. The method of claim 14, comprising, after penetrating the joint capsule, retracting the needle from the joint capsule.

17. The method of claim 14, comprising, after penetrating the joint capsule, inserting the distal portion of the palpating device through the joint capsule.

18. The method of claim 14, wherein extending the needle to penetrate the joint capsule comprises partially extending the needle to partially penetrate the joint capsule, and the method comprises, after partially penetrating the joint capsule, fully extending the needle to fully penetrate the joint capsule.

19. The method of claim 14, wherein inserting the distal portion of the palpating device into the target site comprises inserting a cannula attached to the distal portion of the palpating device into the target site.

20. The method of claim 14, wherein extending the needle of the palpating device comprises orienting a leading tip of the needle that is aligned with a side of the handle comprising the slider.
